(19) United States
(12) Patent Application Publication
(10) Pub. No.: US 2022/0331815 A1
(43) Pub. Date: Oct. 20, 2022

(54) ELECTROSTATIC FILTER UNIT FOR AN AIR CLEANING DEVICE AND AIR CLEANING DEVICE

(71) Applicant: BSH Hausgeräte GmbH, Munich (DE)

(72) Inventors: Georg Hepperle, Heilbronn (DE); Daniel Vollmar, Pfinztal (DE)

(21) Appl. No.: 17/640,363

(22) PCT Filed: Oct. 1, 2020
(86) PCT No.: PCT/EP2020/077528

§ 371 Date: Mar. 4, 2022

(30) Foreign Application Priority Data
Oct. 23, 2019 (DE) 10 2019 216 344.0

(51) Int. Cl. B03C 3/12; B01D 46/10; B01D 46/42; B01D 46/00; B03C 3/82; B03C 3/09; B03C 3/08; B03C 3/41; B03C 3/47; B03C 3/155; B03C 3/66; A61L 9/22; F24C 15/20; F24F 8/192

(52) U.S. Cl. CPC B03C 3/12; B01D 46/10; B01D 46/4209; B01D 46/0032; B03C 3/82; B03C 3/09; B03C 3/08; B03C 3/41; B03C 3/47; B03C 3/155; B03C 3/66; A61L 9/22; F24C 15/2035; F24F 8/194; A61L 2209/14

(57) ABSTRACT

An electrostatic filter unit for an air cleaning device includes an ionization unit for charging a particle contained in an air flow, a separation unit arranged downstream of the ionization unit in a direction of flow of the air, with the separation unit including at least two collecting electrodes which are air-permeable, and a protection element for at least one element of the ionization unit.

The invention relates to a filter unit for an air cleaning device, in particular a vapor extractor, and an air cleaning device having at least one filter unit.

Air cleaning devices can include for example air cleaners for filtering room air, devices for filtering air that is drawn into a passenger compartment of a motor vehicle, or vapor extractors for kitchens, for example in the form of extractor hoods. These air cleaning devices are conventionally provided to filter out liquid and solid impurities, as well as odors, from the impure air or vapors and steam that are produced during cooking. Mechanical filters are usually employed for this purpose. Examples of mechanical filters include expanded metal filters, perforated sheet metal filters, baffle filters (also known as cyclone filters), edge suction filters and porous foamed media. All of these filter media use mechanical separation mechanisms for filtering, for example the diffusion effect, blocking effect and particularly the inertia effect.

When using the inertia effect for the purpose of separation, the particle is not able to follow the streamline of the gas (air) around the individual filter fibers, expanded metal layers, porous media or similar due to its mass inertia, and therefore collides with them. With regard to the dominant inertia effect, the probability of a particle hitting the individual fibers of the filter medium (corresponding to the overall separation efficiency) increases with increasing particle speed, increasing particle diameter, increasing filter packing density and filter thicknesses in the direction of flow, and with decreasing filter fiber diameter of the filter medium.

These filter units have the disadvantage that in particular a high inflow speed must be achieved in order to ensure a satisfactory filter efficiency for particle diameters greater than 1 μm.

In addition to this, DE 2146288 A for example discloses an extractor hood in which an electrostatic filter unit is used. The electrostatic filter unit in this extractor hood consists of plate-form separation electrodes and counter electrodes, and wire-form ionization electrodes. The plate-form separation electrodes and counter electrodes are interconnected via electrically conductive paths and are so arranged that the air entering the filter element initially flows onto the separation electrodes, between which are located wire-form ionization elements, and then arrives at the counter electrodes which are higher up. The separation electrodes are fastened to the housing of the extractor hood via a partition wall. Furthermore, a high-voltage device connected to the electrodes of the filter unit is provided in the vicinity of the suction opening of the housing of the extractor hood.

This filter unit has the disadvantage that it requires considerable structural space and simultaneously presents a danger to the user.

The object of the invention is therefore to provide a solution by means of which it is possible reliably to ensure adequate filter efficiency using a simple structure, while at the same time ensuring safety of use.

According to a first aspect, the object is therefore achieved by an electrostatic filter unit for an air cleaning device, said filter unit comprising an ionization unit and a separation unit with at least two collecting electrodes. The electrostatic filter unit is characterized in that the at least two collecting electrodes are air-permeable and that the filter unit comprises at least one protection element for at least one of the elements of the ionization unit.

The electrostatic filter unit is also referred to in the following as a filter unit or electrostatic filter. The filter unit has an ionization unit and a separation unit. The ionization unit can also be referred to as the ionization zone and the separation unit as the separation zone. In order to allow an electrostatic separation of particles that are present in the air, these must first be electrostatically charged (ionized). The ionization unit is used for this purpose. The separation unit is connected downstream of the ionization unit in the direction of flow. The ionization unit preferably has at least one ionization electrode and at least one counter electrode. Voltage (preferably high voltage) is applied to the ionization electrode. When impure air flows through the ionization unit, solid and liquid particles are electrostatically (electrically) charged by the ionization electrode, which can also be referred to as the emission electrode, by means of a corona discharge. The ionization electrode, which can take the form of a wire ionization electrode, is preferably arranged between two plate-form counter electrodes in the ionization unit. This is necessary because the particles have generally no electrical charge in their original state or have insufficient electrical charge for efficient electrostatic separation. The purpose of the ionization unit is the electrical charging of each individual particle until it reaches its maximum electrical saturation charge.

The separation unit comprises at least two collecting electrodes. The at least two collecting electrodes are preferably arranged parallel to each other. The at least two collecting electrodes are exposed to an electrical high voltage, thereby together forming an electrical field. In this context, at least one collecting electrode is a negative collecting electrode and at least one collecting electrode is a positive collecting electrode. The level or magnitude of the electrical field strength in this case is largely dependent on the electrical potential, i.e. on the magnitude of the voltage in kV, the distance between the positive and negative collecting electrodes, and the geometric form of the collecting electrodes.

The air with the electrically charged particles emerging from the ionization unit flows into the separation unit. As a result of the electrical field that is formed there between the collecting electrodes, the particles are separated at the collecting electrodes and thereby filtered out of the air.

For the purpose of ionizing the particles in the air and for the separation thereof, an electrical high voltage of several thousand volts is required, it being possible to apply both positive and negative high voltage in this case. For the purpose of generating/producing this required electrical high voltage, use is made of a high-voltage transformer, which can also be referred to as a high-voltage generator or a high-voltage power supply. This high-voltage transformer supplies the ionization zone and the separation zone of the electrostatic filter cartridge with electrical high voltage or electrical energy. The high-voltage transformer in this case is preferably implemented in the electrostatic filter unit, which can also be referred to as a filter module or filter cartridge. The electrostatic filter module/cartridge is preferably arranged in the air intake zone of the vapor extractor in order to protect components of the vapor extractor which are situated behind said filter module from cooking steam/aerosols/dirt. However, such a filter device can optionally also be arranged in the vapor stream at the air outlet zone, or along the airflow path between the inlet zone and the outlet zone of the vapor extractor.

According to the invention, the collecting electrodes are air-permeable. The collecting electrodes can consist of an air-permeable material or a non-air-permeable material having at least one air conduction opening. In the first case, the collecting electrodes are also referred to as porous collecting electrodes. The collecting electrodes can all consist of the same air-permeable material. It is however also within the scope of the invention for various collecting electrodes to consist of different materials. Using an air-permeable material for the collecting electrodes has the advantage that the manufacture of the electrostatic filter is simplified since the required air permeability is provided by the material itself. Moreover, in the case of an air-permeable material, the openings in the material have a small dimension whereby efficient separation of particles can be ensured as a result of the mechanical separation effect. According to the embodiment in which the collecting electrode consists of a non-air-permeable material having at least one air conduction opening, it is also possible for only some of the collecting electrodes, for example only the positive or only the negative collecting electrodes, to consist of such a material and for the other collecting electrodes to consist of an air-permeable material. Furthermore, it is also possible for only the first collecting electrode for example, i.e. that facing towards the ionizing unit, to consist of a non-air-permeable material with air conduction openings. The non-air-permeable material can be for example sheet metal. The air conduction openings can be for example holes which are stamped into the sheet metal or created by other means. In particular, the non-air-permeable material with air conduction openings can be expanded metal.

The material of the least one collecting electrode can therefore be for example expanded metal, wire cloth, fibrous material, nonwoven fabric, perforated sheet metal, sintered plastic or foam.

A range of advantages are achieved thereby. Firstly, the airstream can flow not only along the collecting electrodes as per the prior art but also through the collecting electrodes. As a result of the air permeability of the collecting electrodes, these can therefore also serve as mechanical filters. Since the separation unit is situated downstream of the ionization unit in the direction of flow, the particles contained in the air enter the separation unit in an electrically charged state. This means that the separation of the particles at the collecting electrodes is caused by both the mechanical filter effects and the electrical charge, i.e. it is achieved by the electrostatic filter effect.

Conventional mechanical filters have the property that, due to the dominant inertia effect for particle diameters >1 μm, the filter efficiency increases with increasing flow speed. In the case of a purely electrostatic filter, by contrast, the filter efficiency increases with decreasing flow speed because the duration of the particle in the ionization and separation zone therefore increases. The combination provided in the present invention means that the advantages of both the mechanical and the electrostatic filter mechanisms are efficiently utilized.

Furthermore, in the case of conventional electrostatic filters, the filter efficiency is highly dependent on the magnitude of the electrical ionization and separation voltage. In the event that the electronic high-voltage components stop working (power failure) or fail due to a short circuit, no filtering function whatsoever is provided. In the case of the present invention, however, the mechanical filter mechanisms or filter effects are preserved. A total failure of the overall filtering function therefore does not occur.

Finally, as a result of the air permeability of the collecting electrodes, particles can be held at least to some extent in the pores or other air conduction openings of the collecting electrodes.

The electrostatic filter unit according to the invention has at least one protection element for at least one of the elements of the ionization unit. Within the meaning of the invention, a protection element designates an element or a device which protects the user from direct or indirect contact with one of the elements of the ionization unit and/or protects the elements of the ionization unit from mechanical damage.

By virtue of providing at least one such protection element, it is possible to operate the electrostatic filter unit at high voltage and thereby achieve the increase in filter efficiency, and to protect both the user and the filter unit.

According to an embodiment variant, the protection element takes the form of a protection element against the effect of force. In the case of this embodiment variant, the protection element can preferably take the form of a plane element which is arranged upstream of the ionization unit in the direction of flow. The protection element is air-permeable in this case.

Additionally or alternatively, the protection element can be a screening element for electrical screening. In particular, the ionization unit and separation unit can be screened.

According to a preferred embodiment variant, the protection element takes the form of an intervention protection device which has at least one layer and is connected upstream of the ionization unit in the direction of flow. By virtue of the protection element being connected upstream of the ionization unit in the direction of flow, it is possible both to prevent physical contact with the ionization unit by the user and to ensure electrical screening of the ionization unit from the user. The size of the intervention protection device is preferably dimensioned such that the side of the ionization unit facing the direction of flow is completely covered by the layer of the intervention protection device.

According to a preferred embodiment variant, at least one layer of the intervention protection device takes the form of a prefilter. A prefilter is a layer which serves to filter out relatively large particles of dirt and dust, in particular particles having a diameter of D_{hyd}>=1 μm. By virtue of the protection element being embodied as a prefilter, in addition to preventing the intervention of the user in the ionization unit and preferably electrically screening the ionization unit and the separation unit, the filter efficiency of the electrostatic filter unit can be increased. The prefilter can consist of expanded metal, perforated sheet metal, wire cloth, welded mesh or woven wire netting.

According to an embodiment variant, the intervention protection device has at least one protection layer and a spacer element in addition to the prefilter. The spacer element is arranged between the prefilter and the protection layer in this case. The protection layer is preferably the layer which is the first layer of the intervention protection device in the direction of flow. The protection layer can be made from an electrically conductive material, in particular metal, or from an electrically insulating material, in particular plastic.

According to an embodiment variant, the protection element consists at least partially of an electrically conductive or antistatic element having a surface resistance R<=10¹¹ Ohms. At least the prefilter is particularly preferably composed of such a material. By virtue of using such material, any voltage that is present in the prefilter can at least be discharged.

According to a further embodiment variant, at least one protection element takes the form of at least part of a housing for the ionization unit. In particular, the protection element in this embodiment preferably takes the form of at least part of an insulation housing of the ionization unit. In this embodiment variant, the protection element is preferably made from an insulating material. By virtue of providing an insulating protection element, it is possible in particular to accommodate voltage-charged elements of the ionization unit in such a way that they cannot be accessed by the user.

According to an embodiment variant, the filter unit has a high-voltage transformer and the high-voltage transformer is integrated in one of the housing parts. It is thereby possible to prevent access to the high-voltage transformer by the user. It is also possible by integrating the high-voltage transformer into the filter unit to simplify the structure of the air cleaning device, since the air cleaning device only needs to have lines and contact points for low voltage.

According to a preferred embodiment variant, the filter unit has a protection element which takes the form of a prefilter and the prefilter is connected to the return of the secondary side of the high-voltage transformer.

According to an embodiment variant, the electrostatic filter unit has a filter frame and the filter frame is connected to the return of the secondary side of the high-voltage transformer.

According to an embodiment variant, the collecting electrodes lie perpendicular to the direction of flow of the air through the ionization unit. The direction of flow of the air through the ionization unit is preferably parallel to the counter electrode or counter electrodes of the ionization unit. This means that the collecting electrodes preferably lie transversely and in particular perpendicular to the counter electrode or counter electrodes of the ionization unit.

By virtue of this orientation of the air-permeable collecting electrodes, the entire airstream entering the separation unit can be routed through the collecting electrodes. The filter efficiency is therefore increased further. Furthermore, the structural space required for the filter unit can be minimized by virtue of this orientation of the collecting electrodes. In contrast with filter units in which the collecting electrodes lie parallel to the airstream and preferably parallel to the counter electrode or counter electrodes of the ionization unit, the height or length of this embodiment of the filter unit according to the invention is smaller because the collecting electrodes lie transversely in this direction.

According to an embodiment variant, the collecting electrodes are contiguously disposed. In the case of conventional electrostatic filters with plate separators or tube separators in the separation unit, considerably more space is required in relative terms for the overall electrostatic filter and specifically for the separation zone. In the case of the filter unit according to the invention, the collecting electrodes are air-permeable. The collecting electrodes preferably take the form of air-permeable plates or layers. Consequently, even providing for a plurality of collecting electrodes disposed one above the other, the overall height of the stack of collecting electrodes is small. Furthermore, by virtue of the small distance of the collecting electrodes from each other, the particulate material which is separated/filtered between the individual air-permeable collecting electrodes can be retained between the collecting electrodes due to capillary action. This means that the separation zone according to the invention can itself store these particles, in addition to storage in the collecting electrodes.

According to the invention, the sequence of the collecting electrodes in the separation unit is freely selectable. For example, it is possible to arrange a positive collecting electrode on that side of the separation unit which faces towards the ionization unit and via which air enters the separation unit, and then arrange negative and positive collecting electrodes alternately in each case. Alternatively, a negative collecting electrode can be arranged as a first collecting electrode at the side facing towards the ionization unit, followed by positive and negative collecting electrodes alternately.

According to a further embodiment variant, it is however also possible for at least two adjacent collecting electrodes to have the same polarity. This means that, for example, two or more negative collecting electrodes can be arranged between two positive collecting electrodes.

According to a preferred embodiment variant, at least one of the collecting electrodes has an insulation coating. The insulation coating can be deposited onto the collecting electrodes by means of powder coating, dipping or other coating method. In this context, the respective collecting electrode is preferably completely electrically insulated, the insulation coating being left open at the respective electrical contact points that are required in order to apply voltage to the collecting electrode. It is thereby possible to prevent electrical short circuits and associated voltage dips between the individual alternating live collecting electrodes.

According to the invention, all collecting electrodes of the separation unit can have an insulation coating. However, provision is preferably made for electrically insulating only the positive collecting electrodes. The particles are charged in the ionization unit. If a positively charged particle strikes a negative collecting electrode, it should also release its charge again, since the electrical field between the layers would otherwise be weakened thereby over time. However, by virtue of the positive collecting electrodes having an insulation coating in the cited embodiment variant, an electrical short circuit between the positive and negative collecting electrodes can be prevented even if the collecting electrodes are a small distance apart or contiguously disposed.

According to a further aspect, the invention relates to an air cleaning device which has at least one electrostatic filter unit according to the invention.

Advantages and features that are described in relation to the electrostatic filter unit are correspondingly valid (if applicable) in relation to the air cleaning device and vice versa.

The air cleaning device can be for example an air cleaner for filtering room air, a device for filtering air that is drawn into a passenger compartment of a motor vehicle, or a vapor extractor for kitchens. According to the invention, the air cleaning device can have a plurality of electrostatic filter units according to the invention. The at least one electrostatic filter unit is preferably arranged on the suction side of the air cleaning device. It is however also within the scope of the invention additionally or alternatively to provide at least one electrostatic filter unit on the air outlet side of the air cleaning device.

According to an embodiment variant, the air cleaning device takes the form of an extractor hood and the extractor hood has at least one contact point for the contact with a high-voltage transformer of the filter unit.

The invention is described again in the following with reference to the appended figures, in which:

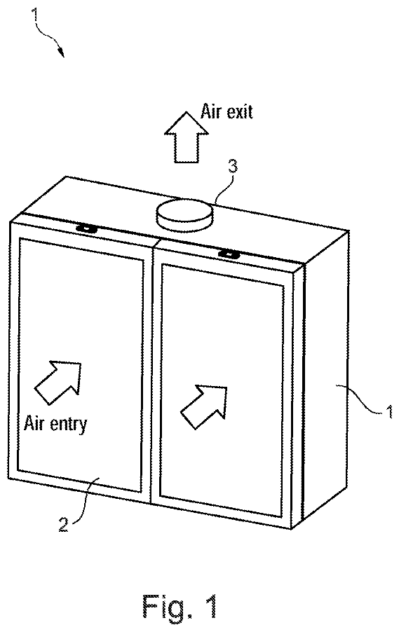

FIG. 1 shows a schematic perspective view of an embodiment of an air cleaning device according to the invention;

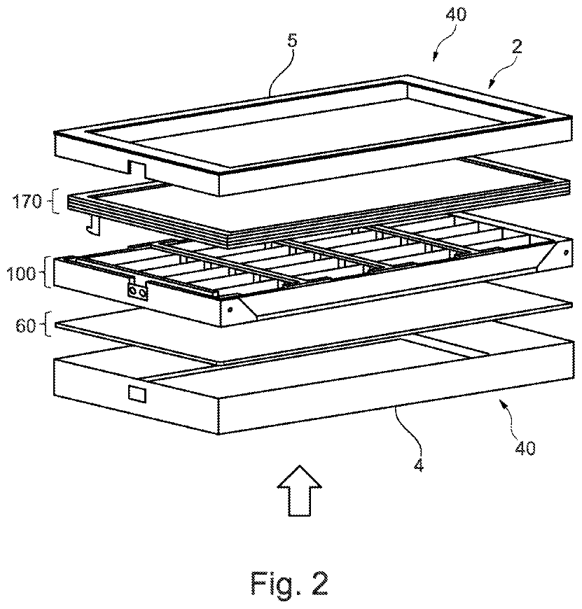

FIG. 2 shows a schematic exploded diagram of an embodiment of an electrostatic filter unit according to the invention;

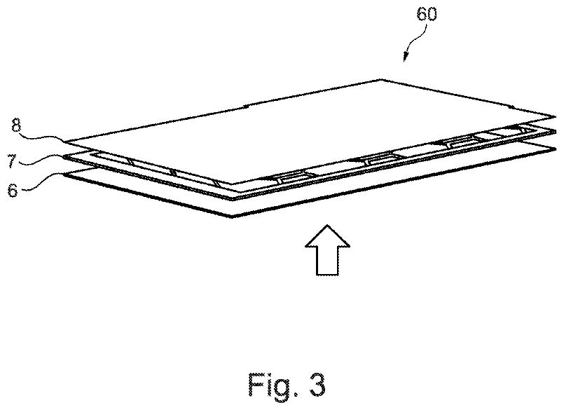

FIG. 3 shows a schematic exploded diagram of the intervention protection device of the electrostatic filter unit according to FIG. 2;

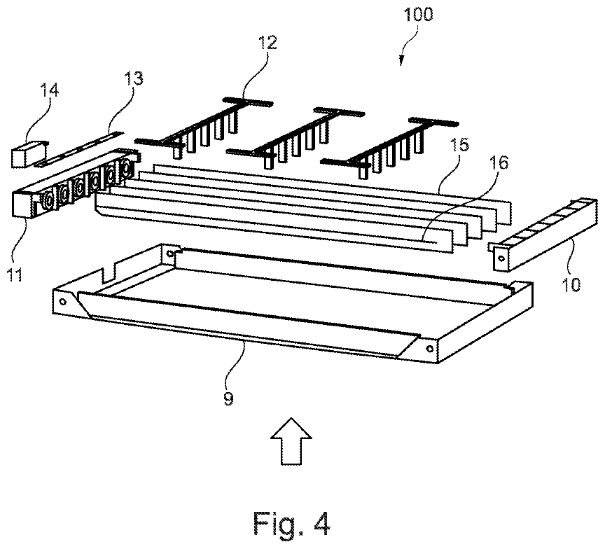

FIG. 4 shows a schematic exploded diagram of the ionization unit of the electrostatic filter unit according to FIG. 2;

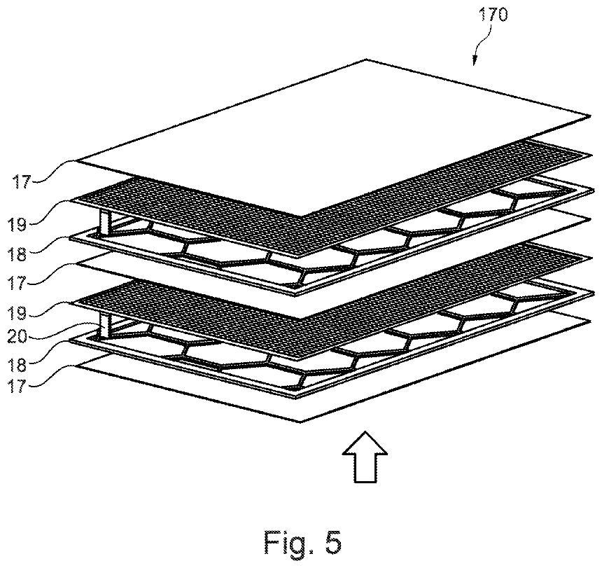

FIG. 5 shows a schematic exploded diagram of the separation unit of the electrostatic filter unit according to FIG. 2;

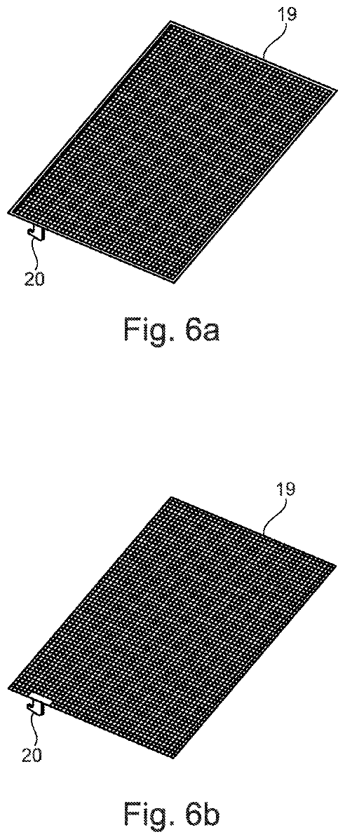

FIGS. 6a and 6b show schematic perspective views of embodiment of the collecting electrodes of the electrostatic filter unit according to FIG. 2;

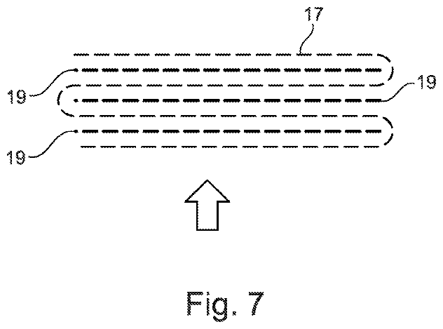

FIG. 7 shows a schematic sectional view of an embodiment of the collecting electrodes of the electrostatic filter unit according to the invention;

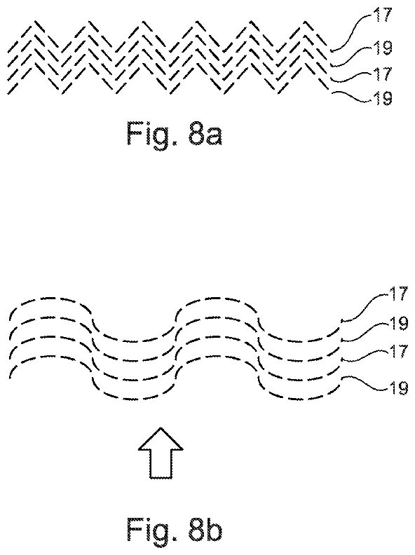

FIGS. 8a and 8b show schematic sectional views of further embodiment of the collecting electrodes of the electrostatic filter unit according to the invention;

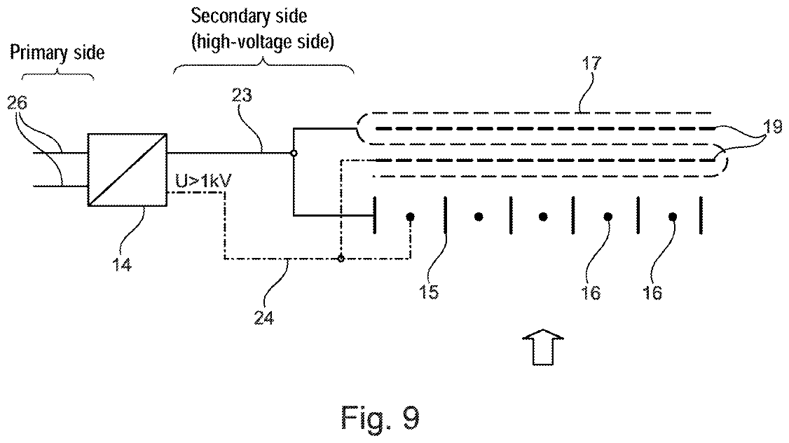

FIG. 9 shows a schematic diagram of the cabling of an embodiment of the electrostatic filter unit according to the invention; and

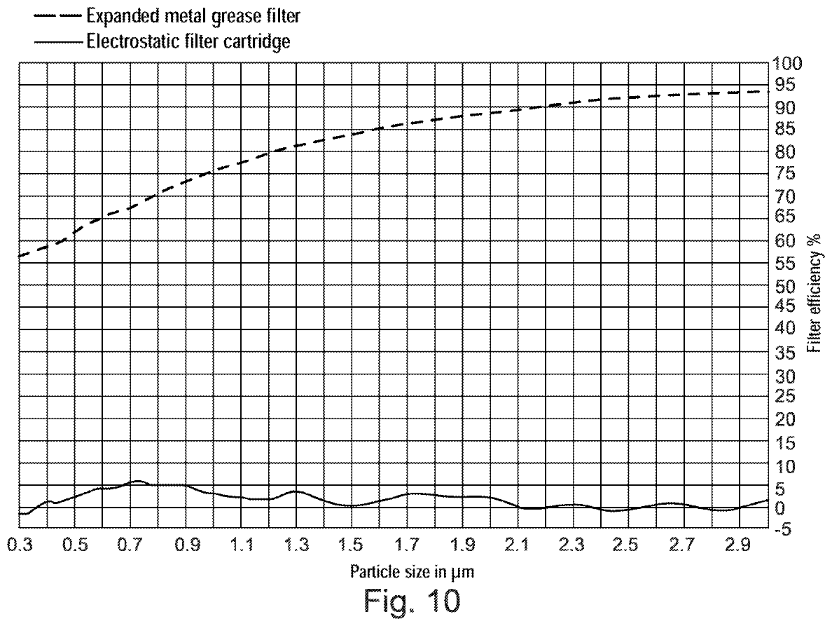

FIG. 10 shows a schematic diagram of the filter efficiency of the electrostatic filter unit according to the invention in comparison with expanded metal filters.

FIG. 1 shows an embodiment of an air cleaning device 1 according to the invention. In the embodiment shown, the air cleaning device 1 takes the form of an extractor hood. In the embodiment shown, a suction opening in which are arranged two electrostatic filter units 2 is formed in the front side of the air cleaning device 1. It is however also within the scope of the invention for only one or more than two electrostatic filter units 2 to be arranged in the suction opening. An air outlet connection piece 3 for the air exit from the air cleaning device 1 is provided in the top side of the air cleaning device. In the embodiment shown, the suction opening and therefore the electrostatic filter units 2 are oriented vertically. It is however also within the scope of the invention for the suction opening and therefore the electrostatic filter units 2 to be oriented in any desired adjustment angle between the vertical and the horizontal. In the embodiment shown, the electrostatic filter units 2 have a cartridge format. The electrostatic filter units 2 are therefore also referred to as filter cartridges. In particular, the electrostatic filter units 2 have a width and length which is greater than their depth in each case. Depth in this context refers to the dimension in the main direction of flow, which is horizontal in the embodiment shown. The main direction of flow is indicated by a block arrow in the figures. The electrostatic filter units 2 cover the suction opening completely. This means that all of the air arriving at the air cleaning device 1 is routed through the electrostatic filter units 2. During operation of the air cleaning device, the impure air flows through the planar surface areas of the electrostatic filter units 2 and by means of the electrostatic and mechanical filter mechanisms is purified of liquid and solid impurities/particles, in particular those having a hydraulic diameter D_{hyd}>=0.001 μm, as a result of filtering. When the electrostatic filter units 2 have reached their saturation level in respect of impurities, they can be removed from the air cleaning device and cleaned in the dishwasher or manually by hand using water and detergent.

FIG. 2 shows a schematic exploded diagram of an embodiment of an electrostatic filter unit 2 according to the invention. In the embodiment shown, the electrostatic filter unit 2 consists of a filter frame 40, an intervention protection device 60, an ionization unit 100 and a separation unit 170. These individual function modules of the electrostatic filter unit 2 have a plane format or define a surface area in each case. In the embodiment shown, the filter frame 40 consists of two parts. That part shown as the lower part in FIG. 2 is subsequently referred to as the filter frame front side 4 and that part of the filter frame 40 shown as the upper part is referred to as the filter frame rear side 5. For the purpose of manufacturing or assembling the electrostatic filter unit 2, the intervention protection device 60, the ionization unit 100 and the separation unit 170 can be placed into the filter frame front side 4 and then closed off using the filter frame rear side 5.

FIG. 3 shows the embodiment of the intervention protection device 60 in an exploded diagram. According to the present invention, the intervention protection device 60 forms a protection element. In the embodiment shown, the intervention protection device 60 consists of three individual parts. In particular, the intervention protection device 60 consists of a protection layer 6, a spacer element for intervention protection 7 and a filter layer for prefiltering 8, also referred to as a prefilter in the following. The individual parts of the intervention protection device 60 have a plane format or layer format in each case. The first layer, which forms the actual protection layer 6, protects the individual parts of the intervention protection device 60, and modules of the electrostatic filter unit 2 that are situated behind said first layer, from the effects of any improper external force. Furthermore, the protection layer 6 can enhance the visual effect of the overall filter cartridge as a design element. The protection layer 6 can be made from an electrically conductive material, in particular metal, or an electrically insulating material, in particular plastic.

In addition to the mechanical protection from the effect of force, the intervention protection device 60 can also serve as an electrical protection element. In particular, the filter layer for prefiltering 8 serves as an electrical protection element. The filter layer for prefiltering 8 serves firstly as a prefilter for relatively large particles of dirt and dust, in particular for particles where D_{hyd}>=1 μm. Secondly, the filter layer for prefiltering 8 also serves as an electrical screen. In this context, the screening by the filter layer for prefiltering 8 takes place in accordance with the principle of a Faraday cage. In particular, the ionization unit 100 and the separation unit 170, which are situated behind the filter layer for prefiltering 8 in the direction of flow, are screened thereby. In particular, the filter layer for prefiltering 8 has the effect that the external intake zone, i.e. the zone in front of the electrostatic filter unit 2 in the direction of flow, remains free of fields owing to the static and quasistatic electrical fields of the ionization unit, and the resulting outward electrical influence, in particular beyond the protection layer 6, is prevented. Unwanted static charging of the protection layer 6 is thereby prevented. The filter layer for prefiltering 8 preferably consists of an air-permeable electrically conductive or antistatic material. For example, the filter layer for prefiltering can be manufactured from electrically conductive plastic. The filter layer for prefiltering 8 can however also consist of for example expanded metal, perforated sheet metal, wire cloth, welded mesh or woven wire netting. The filter layer for prefiltering 8 preferably has a surface resistance R<=10¹¹ Ohms. In the embodiment shown, the filter layer for prefiltering 8 consists of only one layer. It is however also within the scope of the invention for the filter layer for prefiltering 8 to consist of more than one layer.

A further layer of the intervention protection device 60 is provided between the filter layer for prefiltering 8 and the protection layer 6. This is referred to as a spacer element for intervention protection 7 and serves to keep the filter layer for prefiltering 8 and the protection layer 6 at a defined distance from each other.

FIG. 4 shows an exploded view of an embodiment of the ionization unit 100 of the electrostatic filter unit 2. In the embodiment shown, the ionization unit 100 consists of an inner frame 9, two insulation housing parts 10, 11, counter electrodes 15 and ionization elements 16 which can also be referred to as emission electrodes. In the embodiment shown, provision is also made for spacers 12 which keep the counter electrodes 15 at a distance from each other. In the embodiment shown, the ionization unit 100 further comprises a high-voltage transformer 14 and a high-voltage bus bar 13.

The insulation housing parts 10 and 11 are inserted into the inner frame 9. The insulation housing parts 10, 11 each have the format of a rectangular bar and are inserted into the inner frame 9 at opposite ends, in particular longitudinal ends thereof. The insulation housing parts 10, 11 each have contact openings on one side in which the ionization elements 16 are placed. The insulation housing parts 10 and 11 are inserted into the inner frame 9 in such a way that the contact openings face towards each other. This means that the ionization elements 16, which can take the form of wires, can be suspended between the insulation housing parts 10, 11. The counter electrodes 15, which are formed by plates in the embodiment shown, are slotted into the inner frame 9 and preferably also into the insulation housing parts 10, 11. The spacers 12 are also installed, keeping the counter electrodes 15 at a distance from each other along their entire length. The insulation housing parts, 10, 11 preferably consist of an electrically insulating material and preferably have a surface resistance R>=10¹¹ Ohms. The insulation housing parts 10, 11 preferably consist of ceramic or plastic.

In one of the insulation housing parts 10, 11 in the embodiment shown, a mounting frame is also provided in the insulation housing part 11. In the mounting frame are provided the high-voltage transformer 14, the high-voltage bus bar 13 and various cabling elements and contacts (not shown) which are required for the electrical contacting of the relevant individual parts. The ionization elements 16 are then secured between the insulation housing parts 10, 11 and in particular between the high-voltage bus bar 13 of the insulation housing part 11 and the insulation housing part 10.

FIG. 5 shows an embodiment of the separation unit 170 of the electrostatic filter unit 2. The separation unit 170 is constructed from negative collecting electrodes 17, separation unit spacer elements 18 and positive collecting electrodes 19. The spacer elements 18 keep the positive collecting electrodes 19 and negative collecting electrodes 17 at a defined distance from each other. The positive collecting electrodes 19 are preferably exposed to a high voltage U>1 kV DC (direct current). The positive collecting electrodes 19 are preferably completely electrically insulated with an electrically insulating cover as far as the contact points 20, in order to prevent electric arcing and short circuits. For the purpose of insulation, the positive collecting electrodes 19 can be coated. Possible coating methods include functional powder coating, fluidized-bed coating, sol-gel, dip-coating, enameling or painting. An electrically conductive material, in particular metal, is preferably used as a base material or substrate for the positive collecting electrodes 19. Particularly preferably, the materials shown in FIGS. 6a and 6b can be used as a substrate for the positive collecting electrodes 19. In the embodiment variant according to FIG. 6a, the substrate takes the form of perforated sheet metal or expanded metal, and according to FIG. 6b a welded mesh.

As an alternative to metal, it is also possible to use electrically conductive or antistatic plastics having a surface resistance R<=10¹¹ Ohms for the substrate of the positive collecting electrodes 19.

An air-permeable medium is likewise used for the negative collecting electrodes 17. The negative collecting electrodes 17 also consist of an electrically conductive material, for example metal, or an antistatic material, for example plastic having a surface resistance R<=10¹¹ Ohms. For example, expanded metal, perforated sheet metal, wire cloth, welded mesh, woven wire netting and plastic mesh can be used for the negative collecting electrodes 17.

FIG. 7 shows an alternative embodiment of the collecting electrodes 17, 19 of the separation unit 170. In the case of the embodiment shown in FIG. 7, the negative collecting electrode 17 consists of a single part which is disposed between the positive collecting electrodes 19 in a snaking or meandering manner. This embodiment has the advantage that the individual negative collecting electrodes 17 do not additionally need to be electrically contacted to each other via contacts, etc.

FIGS. 8a and 8b show further embodiment of the collecting electrodes of the separation unit 170. In these embodiment variants, the collecting electrodes 17, 19 have a pleated or wavy structure respectively. This measure ensures both a reduction in the loss of pressure Delta p [Pa] of the airstream flowing through the filter medium (the separation unit 170) and an increase in the filter efficiency assuming outline conditions are otherwise unchanged. In particular, the surface area of the collecting electrodes 17, 19 can be increased for the same width and length of the filter frame 40.

In each of the embodiment of the collecting electrodes 17, 19, these can consist of a single layer. Alternatively, the positive collecting electrode 19 can also be constructed from a plurality of layers n>=1. The same applies to the negative collecting electrode 17 likewise.

The number of positive collecting electrodes 19 per filter module is >=1. The number of negative collecting electrodes 17 per filter module is likewise >=1. Spacer elements 18 can be used to increase the filter efficiency. Alternatively, these can be omitted and the positive and negative collecting electrodes 17, 19 stacked directly on top of each other. However, the spacer elements 18 result in a performance increase of the filter efficiency.

The sequence of the positive and negative collecting electrodes 17, 19 is preferably alternating as shown by way of example in FIG. 5. In addition, the first and last collecting electrode in the direction of flow can be either a positive or a negative collecting electrode in each case. This means that the first layer or electrode can be positive or negative and the last layer or electrode can likewise be positive or negative.

FIG. 9 schematically shows the electrical cabling of the electrostatic filter unit 2. The electrical power supply using extra-low voltage (<=50 V AC; <=120 V DC) or low voltage (<=1000 V AC; <=1500 V DC) is provided via contact points or low-voltage connection interfaces 26 on the primary side of the high-voltage transformer 14.

The power supply is provided via contact elements (not shown) between each individual electrostatic filter unit 2 and the vapor extractor or air cleaning device 1. The electrical contacting ideally takes place between the bearing surface of the filter frame rear side 5 and the air cleaning device 1 in the region of the suction opening. The bearing surface of the filter frame rear side 5 is that surface area of the filter frame rear side 5 which is oriented towards the top in FIG. 2. Alternatively, the electrical contact interface can be provided at any desired point of the exterior surface of the filter frame elements 4 and 5.

On the secondary side, the high-voltage line 24 with U>1 kV DC is connected via the high-voltage bus bar 13 (see FIG. 4) to the ionization elements 16 and to all positive collecting electrodes 19. The return line 23 of the secondary side is connected to all counter electrodes 15 and to the negative collecting electrode/electrodes 17. The filter frame front side 4, the filter frame rear side 5 and filter layer for prefiltering 8 are likewise connected to the return line 23 and are on the same electrical potential.

FIG. 10 shows the filter efficiency of an expanded metal filter and an embodiment of the electrostatic filter unit according to the invention 2 for the particle size range 0.3<=D_{hyd}<=3 μm and an average flow speed c<1 m/s. It can be seen from the diagram that the electrostatic filter unit 2 according to the invention has a significantly better filter efficiency than the conventional expanded metal filter of a vapor extractor.

The present invention has a range of advantages.

The completely closed-off electrically conductive or antistatic covering with a preferred surface resistance R<=10¹¹ Ohms, which provides the electrical screening, protects the user/consumer and the surrounding area thereof from electrical risk. By virtue of this structural design, all individual parts under high voltage are completely encapsulated by conductive or antistatic elements. Electrostatic charging of the outermost individual parts which the consumer could come into direct contact with, in particular filter frame and intervention protection, is prevented.

By virtue of the present invention having at least one protection element, electrostatic filter mechanisms can be used without presenting any danger to the user. It is thereby possible to achieve significantly better filter efficiency for liquid and solid particles having a hydraulic diameter D_{hyd}<100 μm in comparison with the conventional grease filters in extractor hoods, particularly for low flow speeds c<2 m/s. The improved filter efficiency is achieved in particular in the embodiment of the electrostatic filter unit in which the porous collecting electrodes are used and therefore the existing mechanical separation mechanisms based on diffusion, blocking and inertia effects are supplemented by the electrostatic filter mechanisms. As a result of the significantly improved filter efficiency, particularly in the case of low flow speeds c<2 m/s, those elements of the vapor extractor which are situated behind said filter (fan, housing, odor filter) remain free from oil and dirt accumulations. Particularly in the case of recirculating vapor extractors which have an odor filter (active carbon or zeolite filter) behind the grease filter in the direction of flow, the service life (lifetime) of these recirculating filters is preserved or extended thereby, since they are not contaminated by the finest particles and therefore clogged in this case. Such an electrostatic filter cartridge has a significantly reduced loss of pressure Delta p for the same filter efficiency, in comparison with the conventional grease filter of a vapor extractor,

By virtue of a protection element being provided according to the invention, for example in the form of an insulation housing part, the high-voltage carrier can be implemented in the electrostatic filter unit without this presenting a danger to the user. Since the electrostatic filter unit can also be supplied with electrical energy from the air cleaning device via contacts, using extra-low voltage (<=50 V AC; <=120 V DC) or low voltage (<=1000 V AC; <=1500 V DC), the electrical risk to the user/consumer is further reduced.

Finally, the preferred use of air-permeable “porous” collecting electrodes instead of plate packets (a plate packet corresponds to the structure of conventional electrostatic filters used for separation) reduces the complexity and cost factor of the overall system because fewer individual parts are employed. The use of an air-permeable (porous) insulating collecting electrode results in a finely meshed electrostatic field with high electrical field strength. It allows a high separation performance in a very limited structural space in comparison with conventional plate-type electrostatic filters based on the Penney principle. As a result of the present invention, this high electrical field strength can be used without any risk to the user.

Furthermore, the use of air-permeable collecting electrodes instead of conventional electrostatic filters with plate separation, the latter having only limited storage capacity due to their structural format, results in the availability of more storage capacity. In conventional electrostatic filters with plate separation, filtered/separated particles are collected on the surface of the non-air-permeable separation plate. In the case of larger accumulations of particles, the retention capability of the surface is exhausted. The filtered/separated particles start to become detached. These are then swept away again, for example by the airstream. As a result of using air-permeable/porous collecting electrodes, holding or storing structures are produced. Any detachment of previously separated particles is greatly reduced.

Finally, by virtue of the preferred provision of an electrically insulating coating for the positive collecting electrodes, the electrodes can be placed directly on top of each other without breaking down the electrical field as a result of electrical creepage currents and spark discharges between positive and negative collecting electrodes. The electrostatic filter unit therefore has better resistance to creepage.

### LIST OF REFERENCE CHARACTERS

1 Air cleaning device
2 Electrostatic filter unit
3 Air outlet connection piece of air cleaning device
40 Filter frame
4 Filter frame front side
5 Filter frame rear side
60 Protection element
6 Protection layer
7 Spacer element for intervention protection
8 Filter layer for prefiltering
9 Inner frame
100 Ionization unit
10 Insulation housing part
11 Insulation housing part incl. mounting frame
12 Spacer
13 High-voltage bus bar
14 High-voltage transformer
15 Counter electrode
16 Ionization element/emission electrode
170 Separation unit
17 Negative collecting electrodes
18 Separation unit spacer element
19 Positive collecting electrode
20 Contact of the positive collecting electrodes
23 Return line of secondary side/connection interface of negative collecting electrode
24 High-voltage connection interface/high-voltage line of secondary side
26 Low-voltage connection interface
D_{hyd}=hydraulic diameter

1-14. (canceled)

15. An electrostatic filter unit for an air cleaning device, said electrostatic filter unit comprising:
an ionization unit for charging a particle contained in an air flow;
a separation unit arranged downstream of the ionization unit in a direction of flow of the air, said separation unit including at least two collecting electrodes, said at least two collecting electrodes being air-permeable; and
a protection element for at least one element of the ionization unit.

16. The electrostatic filter unit of claim 15, wherein the protection element is embodied as a screening element configured to provide protection against an effect of force.

17. The electrostatic filter unit of claim 15, wherein the protection element is embodied as an intervention protection device which includes a layer and is connected upstream of the ionization unit in the direction of flow.

18. The electrostatic filter unit of claim 17, wherein the layer is embodied as a prefilter.

19. The electrostatic filter unit of claim 18, wherein the intervention protection device includes a protection layer and a spacer element, said spacer being arranged between the prefilter and the protection layer.

20. The electrostatic filter unit of claim 15, wherein the protection element includes at least partially an electrically conductive or antistatic element having a surface resistance R<=10¹¹ Ohms.

21. The electrostatic filter unit of claim 15, wherein the protection element is configured to form a part of a housing of the ionization unit.

22. The electrostatic filter unit of claim 21, further comprising a high-voltage transformer integrated in the part of the housing.

23. The electrostatic filter unit of claim 18, further comprising a high-voltage transformer integrated in a part of a housing of the ionization unit, wherein the prefilter is connected to a return of a secondary side of the high-voltage transformer.

24. The electrostatic filter unit of claim 23, further comprising a filter frame connected to the return of the secondary side of the high-voltage transformer.

25. The electrostatic filter unit of claim 15, wherein the at least two collecting electrodes lie perpendicular to the direction of flow of the air through the ionization unit.

26. The electrostatic filter unit of claim 15, wherein at least one of the at least two collecting electrodes has an insulation coating.

27. The electrostatic filter unit of claim 15, wherein one of the at least two collecting electrodes is a positive collecting electrode which has an insulation coating.

28. An air cleaning device, comprising an electrostatic filter unit, said electrostatic filter unit comprising an ionization unit for charging a particle contained in an air flow, a separation unit arranged downstream of the ionization unit in a direction of flow of the air, said separation unit including at least two collecting electrodes, said at least two collecting electrodes being air-permeable, and a protection element for at least one element of the ionization unit.

29. The air cleaning device of claim 28, constructed in the form of an extractor hood, said extractor hood having a contact point for contact to a high-voltage transformer of the filter unit.
